(19) United States
(12) Patent Application Publication
(10) Pub. No.: US 2003/0148939 A1
(43) Pub. Date: Aug. 7, 2003

(54) Compositions and methods related to claudin-7

(76) Inventor: Mariana Nacht, Belmont, MA (US)

Correspondence Address: Elizabeth Lassen, Genzyme Corporation, 15 Pleasant Street Connector, Framingham, MA, 01701-9322, US

(21) Appl. No.: 10/068,486

(22) Filed: Feb. 5, 2002

Related U.S. Application Data
(63) Continuation of application No. PCT/US00/21474, filed on Aug. 7, 2000.
(60) Provisional application No. 60/147,752, filed on Aug. 6, 1999.

(51) Int. Cl. A61K 38/17

(52) U.S. Cl. 514/12

(57) ABSTRACT

This invention provides for methods of modulating angiogenesis and/or endothelial cell proliferation. In particular, applications of reducing or inhibiting angiogenesis, tumor growth, endothelial proliferation by the administration of compositions containing Claudin-7 and biological equivalents thereof. The invention also relates to compositions and methods for treatment for disorders associated with angiogeneis (e.g., cancer).

### CROSS-REFERENCE TO RELATED APPLICATIONS

This application claims priority of U.S. Provisional Application No. 60/147,752, filed, Aug. 6, 1999, the contents of which are hereby incorporated by reference into the present disclosure.

### TECHNICAL FIELD

This invention relates to methods and compositions for the modulation of angiogenesis and/or endothelial cell proliferation and to disorders associated with angiogenesis (e.g., cancer).

### BACKGROUND OF THE INVENTION

Blood vessels are constructed by two processes: vasculogenesis, whereby a primitive vascular network is established during embryogenesis from multipotential mesenchymal progenitors; and angiogenesis, in which preexisting vessels send out capillary sprouts to produce new vessels. Endothelial cells are centrally involved in each process. They migrate, proliferate and then assemble into tubes with tight cell-cell connections to contain the blood (Hanahan, Science 277:48-50 (1997)). Angiogenesis occurs when enzymes, released by endothelial cells, and leukocytes begin to erode the basement membrane, which surrounds the endothelial cells, allowing the endothelial cells to protrude through the membrane. These endothelial cells then begin to migrate in response to angiogenic stimuli, forming off-shoots of the blood vessels, and continue to proliferate until the off-shoots merge with each other to form the new vessels.

Normally angiogenesis occurs in humans and animals in a very limited set of circumstances, such as embryonic development, wound healing, and formation of the corpus luteum, endometrium and placenta. However, aberrant angiogenesis is associated with a number of disorders, including, tumor metastasis. In fact, it is commonly believed that tumor growth is dependent upon angiogenic processes. Thus, the ability to increase or decrease angiogenesis has significant implications for clinical situations, such as wound healing (e.g., graft survival) or cancer therapy, respectively.

### DESCRIPTION OF THE FIGURES

FIG. 1 is a cDNA sequence of Claudin 7.

### SUMMARY OF THE INVENTION

By this invention, Claudin 7 is identified as a gene overexpressed in breast cancer. Using SAGE analysis [Velculescu, V. E., L. Zhang, B. Vogelstein, and K. W. Kinzler. Serial analysis of gene expression. Science. 1995. 270(5235): p. 484-7] combined with cDNA arrays, it was found that Claudin 7 was overexpressed at least 100-fold by 85% of primary tumors examined and greater than 10-fold by all metastatic tumors examined (when compared to expression in normal human mammary epithelial cells). Thus, the expression pattern of Claudin 7 suggests that it plays a role in promoting or maintaining tumorigenesis, perhaps by altering adhesion properties of the cells and/or by allowing the flow of soluble growth factors or angiogenic molecules that are normally prevented from passing. Alternatively, Claudin 7 may create a barrier against angiogenesis inhibitors and/or proteins that hinder cell growth.

Claudin 7 is a Claudins are proteins found in the specialized membrane domain of epithelial and endothelial cells known as tight junctions. In addition to facilitating cell-cell adhesion, tight junctions help maintain cell polarity and serve as a physical barrier to the passing of solutes and water from the paracellular space [Gumbiner, B. M. Breaking through the tight junction barrier. J Cell Biol. 1993. 123(6 Pt 2): p. 1631-3.]. The expression pattern of Claudin-7 suggests that it plays a role in promoting or maintaining tumorigenesis, perhaps by altering adhesion properties of the cells and/or by allowing the flow of soluble growth factors or angiogenic molecules that are normally prevented from passing. Alternatively, Claudin-7 may create a barrier against angiogenesis inhibitors and/or proteins that hinder cell growth. Because Claudin-7 is not expressed in normal breast epithelial cells but is readily detectable in all of the breast tumors examined, it may serve as a good marker of breast carcinogenesis. Claudin 7 may be useful as diagnostic marker or prognostic indicator. In addition, studying the function of Claudin 7 may uncover new pathways for therapeutic intervention.

Prior to the present invention, Claudin 7 was not known to be associated with angiogensis. The present invention is, in one embodiment, drawn to a fragment, derivative or biological equivalent of Claudin 7 that inhibits endothelial cell proliferation, angiogenesis and/or tumor growth in vivo.

In one embodiment, the invention relates to a method of inhibiting tumor growth by delivering or administering a composition comprising a fragment, biological equivalent or derivative of Claudin 7. The composition may further comprise a phsiologically acceptable vehicle.

The invention further relates to a method of inhibiting endothelial cell proliferation comprising delivering or administering a composition comprising a fragment, biological equivalent or derivative of Claudin 7.

Additional features and advantages of the invention will be set forth in the description which follows, and, in part, will be apparent from the description, or may be learned by the practice of the invention. The objectives and other advantages of the invention will be realized and attained by the compounds and methods particularly pointed out in the written description and claims hereof as well as the appended drawings.

### DESCRIPTION OF THE INVENTION

A comparison of gene expression profiles in normal and tumor cells can yield critical information about the processes of transformation and metastasis. While many studies have identified oncogenes and tumor suppressor genes that mark the transformation of cells from the colon, pancreas, and lung, comparable studies in breast cancer have met with limited success. This reflects both the difficulty in finding genetic and epigenetic alterations that are present in a significant proportion of breast cancers, and the heterogeneity of breast cancer itself.

To help elucidate pathways that mediate breast cancer progression, and to find targets for potential therapeutic intervention, we combined SAGE and custom array analysis. We identified differentially expressed genes by generating SAGE profiles of two well characterized breast tumor cell lines, 21PT and 21MT, and two normal mammary epithelial cell cultures. To find genes and ESTs that are consistently expressed at different levels in diseased and normal tissues, a subset of the differentially expressed genes identified by SAGE was then spotted on an array and screened with complex CDNA probes extracted from 17 breast tumor samples and four normal breast epithelial cell cultures. SAGE analysis of 21PT, 21MT and normal breast epithelial cells identified over 200 transcripts that were differentially expressed at least 10-fold between cancer and normal cells. Custom array analysis verified the expression of some of these candidates in primary and metastatic tumors, and identified several differences between the two cancerous states. Our data demonstrates that by coupling the advantages of SAGE and cDNA array differentially expressed genes identified by SAGE was spotted on an array and further screened using clinical tumors as probes. Sixty-eight known genes and ESTs, corresponding to SAGE tags that were either up- or down-regulated at least 5-fold, on average, in 21PT and 21MT with respect to the normal cells, were spotted on replicate nylon membranes. In addition to spotting genes already implicated in breast cancer, genes and ESTs not previously known to be involved in breast cancer were also included on the arrays for further study. SAGE analysis had detected little variation in the averaged expression of EF-1, □-actin, □-tubulin and cyclophilin in the tumor and normal samples, and these genes were included on the arrays as controls. Messenger RNA was extracted from 7 primary breast tumors, 10 metastatic breast tumors, and 4 normal human mammary epithelial cell cultures. The transcripts were converted to ³²P-labeled cDNAs and were used to screen replicate membranes. The signals from each hybridization were quantitated and normalized to decrease variations due to differences in overall hybridization efficiency. For each spotted cDNA target, the average signal intensity was calculated for hybridizations with the primary tumors, metastatic tumors, and normal breast tissue. Array expression profiles for the primary and metastatic tumors were compared to the array expression profiles for the normal breast cells.

As expected from the SAGE results, EF-1, □-actin, □-tubulin and cyclophilin showed relatively even expression by array analysis. The average of the tumor versus normal ratios for these four control genes was 1.1.

Overall, the expression patterns of the chosen genes in the primary and metastatic breast tumors were similar. However, only 6 of the top 10 differentially expressed targets were found in both primary and metastatic tumors. Notably, 2 uncharacterized ESTs were among the 10-most differentially expressed genes in the primary tumor analysis, and 4 undefined ESTs were among the 10-most differentially expressed genes in the metastatic tumor analysis. As expected, MUC-1, HER2/neu and Zn-a-GP were overexpressed in the tumors.

SAGE had detected the tag for the Claudin-7 gene 12 times in the tumor cell lines, whereas no Claudin-7 tags were found in the normal breast cultures. Accordingly, the array analysis showed that Claudin-7 was nearly undetectable in the normal cells but was highly induced in both primary and metastatic tumors. Claudin-7 is a new member of the multigene claudin family [Morita, K., M. Furuse, K. Fujimoto, and S. Tsukita. Claudin multigene family encoding four-transmembrane domain protein components of tight junction strands. Proc Natl Acad Sci USA. 1999. 96(2): p. 511-6.], which has not been previously implicated in cancers.

Gene expression patterns in the primary and metastatic tumors were further analyzed by examining the individual expression levels of the most highly differential genes in each of the tumors used to screen the arrays. Although MUC-1 showed an average overexpression of 32-fold in the primary tumors and 23-fold in the metastatic tumors (Table 3A and 3B), 5/7 of the primary tumors and 7/10 of the metastatic tumors actually expressed it more than 100-fold greater than normal cells. Similarly, Claudin-7 was overexpressed more than 100-fold by 6/7 (85%) of the primary tumors and 6/10 (60%) of the metastatic tumors. The induction of HER-2/neu was less than 10-fold in 100% of the primary tumors and 70% of the metastatic tumors.

Claudin 7 can also be synthesized chemically or biologically, such as by cell culture or recombinant technology, or produced transgenically. Similarly, the particular portions and conformations of Claudin 7, which are the subject of this invention, can be isolated from natural sources, produced transgenically, or can be chemically or biologically synthesized. Recombinant techniques known in the art include, but are not limited to, gene amplification from DNA using polymerase chain reaction (PCR), gene amplification from RNA using reverse transcriptase PCR and NASBA (nucleic acid sequence based amplifications).

The invention also relates to a method of enhancing angiogenesis or endothelial cell proliferation comprising administering a composition comprising an effective amount of an antagonist of Claudin 7. For example, this method can be useful in the treatment of abnormal ovulation, menstruation and placentation, and vasculogenesis, such as in tissue repair, wound healing and tissue grafting.

The fragments, derivatives, and biological equivalents of Claudin 7 having anti-angiogenic properties, that inhibit the proliferation of endothelial cells, and/or have anti-tumor activity are described herein. These Claudin 7 fragments, conformations, derivatives and biological equivalents are collectively termed herein “anti-angiogenic Claudin products,” or “anti-proliferative Claudin 7 products.”

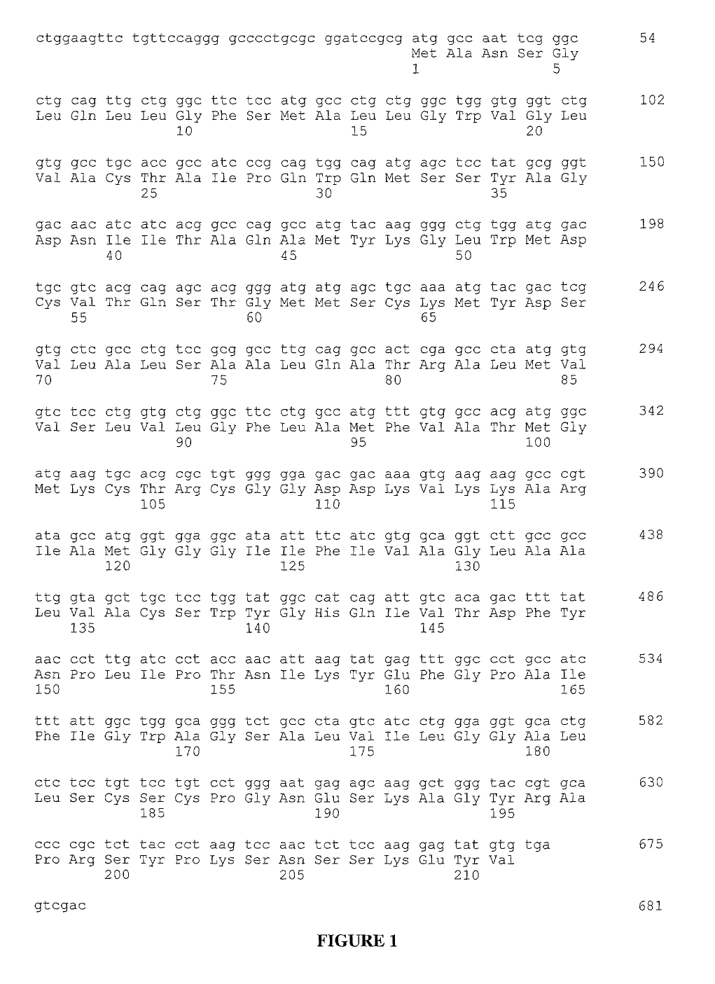

In addition to the sequences of Claudin 7 described above, useful nucleic acid molecules may comprise a nucleotide sequence which is greater than about 80 percent, preferably greater than about 85 percent, more preferably greater than about 90 percent, and even more preferably greater than about 95 percent, identical to the nucleotide sequences of FIG. 1. The substantially identical sequence should, however, retain at least one of the activities of inhibition of endothelial cell proliferation, inhibition of angiogenesis or inhibition of tumor growth (i.e., a biological equivalent).

To determine the percent identity of two nucleotide sequences, the sequences are aligned for optimal comparison purposes (e.g., gaps can be introduced in the sequence of a first nucleotide sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=# of identical positions/total# of positions×100).

The determination of percent identity between two sequences can be accomplished using a mathematical algorithm. A preferred, non-limiting example of a mathematical algorithm utilized for the comparison of two sequences is the algorithm of Karlin et al., Proc. Mad. Acad. Sci. USA, 90:5873-5877 (1993). Such an algorithm is incorporated into the NBLAST program which can be used to identify sequences having the desired identity to nucleotide sequences of the invention. To obtain gapped alignments for comparison purposes, Gapped BLAST can be utilized as described in Altschul et al., Nucleic Acids Res, 25:3389-3402 (1997). When utilizing BLAST and Gapped BLAST programs, the default parameters of the respective programs (e.g., NBLAST) can be used. See http://www.ncbl.nlm.nih.gov. In one embodiment, parameters for sequence comparison can be set at W=12. Parameters can also be varied (e.g., W=5 or W=20). The value “W” determines how many continuous nucleotides must be identical for the program to identify two sequences as containing regions of identity.

As appropriate, nucleic acid molecules of the present invention can be RNA, for example, MRNA, or DNA, such as cDNA and genomic DNA. DNA molecules can be double-stranded or single-stranded; single stranded RNA or DNA can be either the coding, or sense, strand or the non-coding, or antisense, strand. Preferably, the nucleic acid molecule comprises at least about 10 nucleotides, more preferably at least about 50 nucleotides, and even more preferably at least about 200 nucleotides. The nucleic acid molecule can include all or a portion of the coding sequence of a gene and can further comprise additional non-coding sequences such as introns and non-coding 3′ and 5′ sequences (including regulatory sequences, for example). Additionally, the nucleic acid molecule can be fused to a marker sequence, for example, a sequence which encodes a polypeptide to assist in isolation or purification of the polypeptide. Such sequences include, but are not limited to, those which encode a glutathione-S-transferase (GST) fusion protein and those which encode a hernaglutin A (HA) polypeptide marker from influenza.

As used herein, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. In some instances, the isolated material will form part of a composition (for example, a crude extract containing other substances), buffer system or, reagent mix. In other circumstance, the material may be purified to essential homogeneity, for example as determined by PAGE or column chromatography such as HPLC. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Recombinant DNA contained in a vector is included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules. Such isolated nucleic acid molecules are useful in the manufacture of the encoded protein, as probes for isolating homologous sequences (e.g., from other mammalian species), for gene mapping (e.g., by in situ hybridization with chromosomes), or for detecting expression of the gene in tissue (e.g., human tissue) such as by Northern blot analysis.

Thus, DNA molecules which comprise a sequence which is different from the naturally-occurring nucleic acid molecule, but which, due to the degeneracy of the genetic code, encode a substantially similar protein or polypeptide are useful in this invention. The invention also encompasses variations of the nucleic acid molecules of the invention, such as those encoding portions, analogues or derivatives of the encoded protein or polypeptide. Such variations can be naturally-occurring, such as in the case of allelic variation, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably, the nucleotide variations are silent; that is, they do not alter the characteristics or activity of the encoded protein or polypeptide (i.e., a biological equivalent). As used herein, activities of the encoded protein or polypeptide include, but are not limited to, inhibition of angiogenesis, inhibition of endothelial cell proliferation and inhibition of tumor growth. The invention also encompasses sequences that are not identical to Claudin 7.

The invention also pertains to nucleic acid molecules which hybridize under high stringency hybridization conditions (e.g., for selective hybridization) to a nucleotide sequence described herein. Hybridization probes are oligonucleotides which bind in a base-specific manner to a complementary strand of nucleic acid. Such probes include polypeptide nucleic acids, as described in Nielsen et al., Science 254, 14971500 (1991). Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 60%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (1998)) the teachings of which are hereby incorporated by reference. Equivalent conditions can be determined by varying one or more of the parameters given as an example, as known in the art, while maintaining a similar degree of identity or similarity between the target nucleic acid molecule and the primer or probe used. Hybridizable nucleic acid molecules are useful as probes and primers, e.g., for diagnostic applications.

In addition to substantially full-length polypeptides encoded by nucleic acid molecules described herein, the present invention includes biologically active fragments of Claudin 7 and biological equivalents, or analogs thereof, including organic molecules which simulate the interactions of Claudin 7. Biologically active fragments include any portion of the full-length polypeptide which confers a biological function on the variant gene product, including ligand binding and antibody binding, and particularly including inhibition of endothelial cell proliferation, angiogenesis or tumor growth.

Also of use in the invention are fragments or portions of the isolated nucleic acid molecules described above. The term “fragment” is intended to encompass a portion of a nucleic acid molecule described herein which is from at least about 7 contiguous nucleotides to at least about 25 contiguous nucleotides or longer in length. Such fragments are useful as probes, e.g., for diagnostic methods, and also as primers. The nucleotide sequences may also be an isolated portion of any of the nucleotide sequences of Claudin 7, which portion is sufficient in length to distinctly characterize the sequence. Particularly preferred primers and probes selectively hybridize to the nucleotide sequences of Claudin 7. For example, fragments which encode antigenic proteins or polypeptides described herein are useful.

Also within the practice of the invention are anti-angiogenic Claudin 7 products and anti-proliferative Claudin 7 products that are intended to encompass any fragments or conformations of Claudin 7 which have anti-angiogenic and/or anti-proliferative activity, respectively. Anti-angiogenic activity and anti-proliferative activity can be assessed according to methods described herein or according to other methods known in the art or may be any fragments or biological equivalents that mimic the active site.

The biological equivalents of Claudin 7, may include, but are not limited to, fragments of Claudin 7 that comprise the active site; synthetic compounds that mimic the active site; conformational variations of other serpins; other conformations of Claudin 7, aggregate forms and fusion proteins that exhibit anti-angiogenic and anti-proliferative properties.

This invention also pertains to an isolated protein or polypeptide encoded by the nucleic acid molecules of the invention. The encoded proteins or polypeptides of the invention can be partially or substantially purified (e.g., purified to homogeneity), and/or are substantially free of other proteins. According to the invention, the amino acid sequence of the polypeptide can be that of the naturally-occurring protein or can comprise alterations therein. Such alterations include conservative or non-conservative amino acid substitutions, additions and deletions of one or more amino acids; however, such alterations should preserve at least one activity of the encoded protein or polypeptide, i.e., the altered or mutant protein should be a biological equivalent of the naturally-occurring protein. The mutation(s) should preferably preserve the endothelial cell proliferative inhibition, angiogenesis inhibition or tumor growth inhibition activities of the native protein or polypeptide. The presence or absence of biological activity or activities can be determined by various functional assays as described herein. For example, glycosylation variants of Claudin 7 are within the scope of the biological equivalents of Claudin 7.

Moreover, amino acids which are essential for the function of the encoded protein or polypeptide can be identified by methods known in the art. Particularly useful methods include identification of conserved amino acids in the family or subfamily, site-directed mutagenesis and alanine-scanning mutagenesis (for example, Cunningham and Wells, Science 244:1081-1085 (1989)), crystallization and nuclear magnetic resonance. The altered polypeptides produced by these methods can be tested for particular biologic activities, including immunogenicity and antigenicity.

Specifically, appropriate amino acid alterations can be made on the basis of several criteria, including hydrophobicity, basic or acidic character, charge, polarity, size, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie et al., Science 247:1306-1310(1990).

For example, conservative amino acid replacements can be those that take place within a family of amino acids that are related in their side chains. Genetically encoded amino acids are generally divided into four families: (1) acidic=aspartate, glutamate; (2) basic=lysine, arginine, histidine; (3) nonpolar—alanine, valine, leucine, isoleucine, proline, phenylalanine, methionine, tryptophan; and (4) uncharged polar—glycine, asparagine, glutamine, cystine, serine, threonine, tyrosine. Phenylalanine, tryptophan and tyrosine are sometimes classified jointly as aromatic amino acids. For example, it is reasonable to expect that an isolated replacement of a leucine with an isoleucine or valine, an aspartate with a glutamate, a threonine with a serine or a similar conservative replacement of an amino acid with a structurally related amino acid will not have a major effect on activity or functionality.

The polypeptides of the present invention can be used to raise antibodies or to elicit an immune response. The polypeptides can also be used as a reagent, e.g., a labeled reagent, in assays to quantitatively determine levels of the protein or a molecule to which it binds (e.g., a receptor or a ligand) in biological fluids. The polypeptides can also be used as markers for tissues in which the corresponding protein is preferentially expressed, either constitutively, during tissue differentiation, or in a diseased state. The polypeptides can be used to isolate a corresponding binding partner, e.g., receptor or ligand, such as, for example, in an interaction trap assay, and to screen for peptide or small molecule antagonists or agonists.

The present invention also relates to antibodies which bind a polypeptide or protein of the invention. For instance, polyclonal and monoclonal antibodies, including non-human and human antibodies, humanized antibodies, chimeric antibodies and antigen-binding fragments thereof (Current Protocols in Immunology, John Wiley & Sons, N.Y. (1994); EP Application 173,494 (Morrison); International Patent Application W086/01533 (Neuberger); and U.S. Pat. No. 5,225,539 (Winters)) which bind to Claudin 7 proteins or polypeptides are within the scope of the invention. A mammal, such as a mouse, rat, hamster or rabbit, can be immunized with an immunogenic form of the protein (e.g., the full length protein or a polypeptide comprising an antigenic fragment of the protein which is capable of eliciting an antibody response). Techniques for conferring immunogenicity on a protein or polypeptide include conjugation to carriers or other techniques well known in the art. The protein or polypeptide can be administered in the presence of an adjuvant. The progress of immunization can be monitored by detection of antibody titers in plasma or serum. Standard ELISA or other immunoassays can be used with the immunogen as antigen to assess the levels of antibody.

As described herein, Claudin 7 and/or a fragment, biological equivalent, or derivative can be made or isolated by numerous methods known in the art, including, but not limited to, purification, transgenic and recombinant methods.

The invention provides expression vectors containing a nucleic acid sequence described herein, operably linked to at least one regulatory sequence. Many such vectors are commercially available, and other suitable vectors can be readily prepared by the skilled artisan. “Operably linked” or “operatively linked” is intended to mean that the nucleic acid molecule is linked to a regulatory sequence in a manner which allows expression of the nucleic acid sequence. Regulatory sequences are art recognized and are selected to produce the encoded polypeptide or protein. Accordingly, the term “regulatory sequence” includes promoters, enhancers, and other expression control elements which are described in Goeddel, Gene Expression Technology. Methods in Enzymology 185, Academic Press, San Diego, Calif. (1990). For example, the native regulatory sequences or regulatory sequences native to the transformed host cell can be employed. It should be understood that the design of the expression vector may depend on such factors as the choice of the host cell to be transformed and/or the type of protein desired to be expressed. For instance, the polypeptides of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance.

Prokaryotic and eukaryotic host cells transfected by the described vectors are also provided by this invention. For instance, cells which can be transfected with the vectors of the present invention include, but are not limited to, bacterial cells such as E. coli (e.g., E. coli K12 strains), Streptomyces, Pseudomonas, Serratia marcescens and Salmonella typhimurium, insect cells (baculovirus), including Drosophila, fungal cells, such as yeast cells, plant cells and mammalian cells, such as thymocytes, Chinese hamster ovary cells (CHO), and COS cells.

In one embodiment, at least one fragment, biological equivalent, or derivative of Claudin 7 that is useful in the practice of the invention is produced in vivo or ex vivo via gene therapy. For example, gene therapy may be used to produce Claudin 7 or a biological equivalent. An enzyme that effectuates a conformational change in Claudin 7 or a biological equivalent to an anti-angiogenic product is then delivered or the biological equivalent. By using tissue specific expression an anti-angiogenic product may be produced in vivo at a desired site.

Thus, a nucleic acid molecule described herein can be used to produce a recombinant form of the protein via microbial or eukaryotic cellular processes. Ligating the polynucleic acid molecule into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant proteins according to the present invention by microbial means or tissue-culture technology. Accordingly, the invention pertains to the production of encoded proteins or polypeptides by recombinant technology.

The proteins and polypeptides of the present invention can be isolated or purified (e.g., to homogeneity) from recombinant cell culture by a variety of processes. These include, but are not limited to, anion or cation exchange chromatography, ethanol precipitation, affinity chromatography and high performance liquid chromatography (HPLC). The particular method used will depend upon the properties of the polypeptide and the selection of the host cell; appropriate methods will be readily apparent to those skilled in the art.

Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:495-497 (1975); Kozbar et al., Immunology Today 4:72 (1983); and Cole et al., Monoclonal Antibodies and Cancer Therapy, Alan R. Liss, Inc., pp. 7796 (1985)). The term “antibody” as used herein is intended to include fragments thereof, such as Fab and F(ab)2. Antibodies described herein can be used to inhibit the activity of the polypeptides and proteins described herein, particularly in vitro and in cell extracts, using methods known in the art. Additionally, such antibodies, in conjunction with a label, such as a radioactive label, can be used to assay for the presence of the expressed protein in a cell from, e.g., a tissue sample, and can be used in an immunoabsorption process, such as an ELISA, to isolate the protein or polypeptide. Tissue samples which can be assayed include human tissues, e.g., differentiated and non-differentiated cells, such as tumor cells. These antibodies are useful in diagnostic assays, or as an active ingredient in a pharmaceutical composition. For example, passive antibody therapy using antibodies which specifically bind Claudin 7 can be used to modulate (inhibit or enhance) endothelial cell proliferative- or angiogenic-dependent processes such as reproduction, wound healing and tissue repair.

The present invention also encompasses the detection of Claudin 7 to determine the diagnosis or prognosis of endothelial cell proliferation related or angiogenesis-related disorders. As used herein, angiogenesis-related disorders include, but are not limited to, cancers, solid tumors, blood born tumors such as leukemias, tumor metastasis, benign tumors such as hemangiomas, acoustic neuromas, neurofibromas, trachomas and pyogenic granulomas, rheumatoid arthritis, psoriasis, ocular anglogenic diseases such as diabetic retinopathy, retinopathy of prematurity, macular degeneration, corneal graft rejection, neovascular glaucoma, retrolental fibroplasia and rubeosis, Osler-Webber Syndrome, myocardial angiogenesis, plaque neovascularization, telangiectasia, hemophiliac joints, angiofibroma and wound granulation. As used herein, endothelial cell proliferation-related disorders include, but are not limited to, intestinal adhesions, atherosclerosis, scleroderma and hypertrophic scars. Compounds described herein can also be used as birth control agents by preventing the neovascularization required for embryo implantation.

The invention also relates to a kit for detecting the presence of fragments, conformations, derivatives, and biological equivalents of Claudin 7. Typically, the kit will comprise primary reagents (e.g., antibodies) capable of detecting the presence of fragments, conformations, derivatives, and biological equivalents in a sample. The kit may also comprise adjunct reagents suitable for detecting binding of the primary reagent to the target.

The present invention also pertains to pharmaceutical compositions comprising fragments, conformations, derivatives, and biological equivalents of Claudin 7 including polypeptides and other compounds described herein. For instance, a polypeptide or protein, or prodrug thereof, of the present invention can be formulated with a physiologically acceptable medium to prepare a pharmaceutical composition. In one embodiment, an anti-angiogenic pharmaceutical composition comprises a purified form of Claudin 7 that reduces angiogenesis. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Formulation of an agent to be administered will vary according to the route of administration selected (e.g., solution, emulsion, capsule). An appropriate composition comprising the agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Phannaceutical Sciences, 17^{th} Edition, Mack Publishing Co., PA, 1985). For inhalation, the agent can be solubilized and loaded into a suitable dispenser for administration (e.g., an atomizer, nebulizer or pressurized aerosol dispenser).

The pharmaceutical compositions of the present invention may also comprise a composition that effectuates a conformational change in a serpin or produces a fragment, derivative, and biological equivalent of Claudin 7 in vivo, for example, by the delivery of an enzyme.

Methods of introduction at the site of treatment include, but are not limited to, intradermal, intramuscular, intra peritoneal, intravenous, rectal, vaginal, intra ocular, topical, subcutaneous, oral and intra nasal. Other suitable methods of introduction can also include gene therapy, rechargeable or biodegradable devices, viral vectors, naked DNA, lipids and slow release polymeric devices. The pharmaceutical compositions of this invention can also be administered as part of a combinatorial therapy with other agents. Nucleic acid sequences of the invention can be used in gene therapy and introduced either in vivo or ex vivo into cells for expression in a mammalian subject. Cells can also be cultured ex vivo in the presence of proteins of the present invention in order to produce a desired effect on such cells. Treated cells can then be introduced in vivo for therapeutic purposes.

The invention can be used to treat a variety of animals. Suitable animals as used herein include mammals, including, but not limited to, primates (e.g., humans), dogs, cats, cows, horses, pigs, sheep, goats and rodents (e.g., rats, mice and hamsters). Appropriate dosages (e.g., those containing an “effective amount”) of a fragment, derivative or biological equivalent of Claudin 7 will depend upon the physical characteristics of the animal to be treated and on the disorder and (progression thereof) to be treated. One of ordinary skill in the art would readily be able to determine what would be an effective amount. The agent can be administered alone or in combination with other agents or treatment regimes, including chemotherapy and radiation. The agent can be administered in multiple or single administrations provided sequentially or simultaneously.

The present invention will now be illustrated by the following Examples, which are not intended to be limiting in any way. The teachings of all references cited herein are incorporated herein by reference in their entirety.

### EXAMPLES

### Example 1

Expression of Claudin 7

To analyze the function of Claudin 7, the full-length cDNA shown in FIG. 1 (Morita, K, Furuse, M, Fijumoto, K, and Tsukita, S. (1999) Claudin multigene family encoding four-transmembrane domain protein components of tight junction strands. Proc. Natl. Acad. Sci. USA, 96:511-516) can be expressed in 2 mammalian expression systems, for ease of purification/identification of the recombinant fusion proteins.

a) pGEX-6p-1 (from Pharmacia Biotech ; Smith, D B (1993) Purification of glutathione-S-transferase fusion proteins. Methods Mol. Cell Biol. 4:220-229). This vector will provide a fusion protein between glutathione-S-transferase and Claudin-7. The fusion protein can be purified using a glutathione column. In addition, antibodies to GST are commercially available for verification of expression. Following expression of the fusion protein, the GST moiety can be proteolytically cleaved to isolate only the Claudin 7 protein.

b) pQE (from Qiagen; Gu, J., Stephenson, C. G., and Iadarola, M. J. (1994) Recombinant proteins attached to a nickel-NTA column: use in affinity purification of antibodies. BioTechniques 17, 257). This vector will provide expression of the Claudin 7 protein containing 6 additional histidine (His) residues at the carboxy-terminus of the protein. The His-tags allow the fusion protein to be purifed on a nickel column.

### Example 2

Angiogenic Assay

To validate angiogenic activity, Claudin 7, a fragment, biological equivalent or derivative of Claudin 7 can be expressed and incubated with human microvascular endothelial cells (HMVECs) or rat aorta cells to determine

a) proliferation of HMVECs (Pike, S. E., Yao, L., Jones, K. D., Cherney, B., Appella, E., Sakaguchi, K., Nakhasi, H., Teruy-Feldstein, J., Wirth, P, Gupta, G., Tosato, G. (1998) Vasostatin, a Calreticulin Fragment, Inhibits Angiogenesis and Suppresses Tumor Growth. J. Exp. Med. 188:2349-2356.)

b) stimulation of HMVEC tube formation (Gho, Y. S., Kleinman, H. K., Sosne, G. (1999) Angiogenic Activity of Human Soluble Intercellular Adhesion Molecule-1. Cancer Res. 59:5128-5132.)

c) migration of endothelial cells (Chen, Z., Fisher, R. J., Riggs, C. W., Rhim, J. S., Lautenberger, J. A. (1997) Inhibition of Vascular Endothelial Growth Factor-induced Endothelial Cell Migration by ETS1 Antisense Oligonucleotides. Cancer Res. 57:2013-2019.)

d) promotion of ex-vivo angiogenesis/sprout formation (Nicosia, R. F., Ottinetti, A. (1990) Modulation of Microvascular Growth and Morphogenesis by Reconstituted Basement Membrane Gel in Three-Dimensional Cultures of Rat Aorta: A Comparative Study of Angiogenesis in Matrigel, Collagen, Fibrin, and Plasma Clot. In Vitro Cell. Dev. Biol. 26:119-128.)

### Example 3

Tumorogenic Activity

To validate tumorogenic activity, Claudin 7, an expression construct encoding a fragment, biological equivalent or derivative of Claudin 7 can be introduced into primary cultures and/or non-transformed cells, either by electroporation or lipofection (Micka B, Trojaneck B, Niemitz S, Lefterova P, Kruopis S, Huhn D, Wittig B, Schadendorf D, Schmidt-Wolf I G. (2000) Comparison of non-viral transfection methods in melanoma cell primary cultures. Cytokine 12:828-833), to determine if growth properties are altered. Specifically, proliferation, cell-doubling time and sensitivity to apoptosis can be examined (Brugarolas J, Chandrasekaran C, Gordon J I, Beach D, Jacks T, Hannon G J. (1995) Radiation-induced cell cycle arrest compromised by p21 deficiency. Nature 377:552-7.)

What is claimed is:

1. A method of modulating angiogenesis and/or endothelial cell proliferation in a mammal comprising delivery to said mammal a composition comprising at least on fragment, biological equivalent or derivative of Claudin-7, wherein said fragment, biological equivalent or derivative of Claudin-7 modulates angiogensis and/or endothelial cell proliferation.

2. A method of modulating angiogeneis and/or endothelial cell proliferation in a mammal according to claim 1 wherein said composition further comprises a physiologically acceptable vehicle.

3. A method of reducing angiogenesis and/or endothelial cell proliferation in a mammal comprising delivery to said mammal a composition comprising at least on fragment, biological equivalent or derivative of Claudin-7, wherein said fragment, biological equivalent or derivative of Claudin-7 reduces angiogensis and/or endothelial cell proliferation.

4. A method of reducing angiogeneis and/or endothelial cell proliferation in a mammal according to claim 3 wherein said composition further comprises a physiologically acceptable vehicle.

5. A method of reducing tumor growth in a mammal comprising delivery to said mammal a composition comprising at least on fragment, biological equivalent or derivative of Claudin-7, wherein said fragment, biological equivalent or derivative of Claudin-7 reduces tumor growth.

6. A method of reducing tumor growth in a mammal according to claim 5 wherein said composition further comprises a physiologically acceptable vehicle.


SEQUENCE LISTING (2 sequences)

SEQ ID NO: 1 - DNA, 681 bp, Homo sapiens
  CDS (40)..(672)
ctggaagttctgttccaggggcccctgcgcggatccgcgatggccaattggggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccgccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctgggaatgagagcaaggctgggtaccgtgcaccccgctcttaccctaagtccaactcttccaaggagtatgtgtgagtcgac

SEQ ID NO: 2 - protein, 211 aa, Homo sapiens
MANWGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLAALVACSWYGHQIVTDFYNPLIPTNIKYEFGPAIFIGWAGSALVILGGALLSCSCPGNESKAGYRAPRSYPKSNSSKEYV